(19) United States
(12) Patent Application Publication
(10) Pub. No.: US 2021/0043322 A1
(43) Pub. Date: Feb. 11, 2021

(54) System, Method and computer program product to evaluate, communicate, record, and transact patient related data for monitoring user, providing resources and completing procedures in abating drug abuse.

(71) Applicant: Arya Deepak Keni, Woodbridge, NJ (US)

(72) Inventor: Arya Deepak Keni, Woodbridge, NJ (US)

(73) Assignee: Arya Deepak Keni, Woodbridge, NJ (US)

(21) Appl. No.: 16/537,584

(22) Filed: Aug. 11, 2019

(51) Int. Cl. G16H 50/20; G06F 16/245; G16H 10/60; G16H 20/00; G06N 20/00

(52) U.S. Cl. CPC G16H 50/20; G06F 16/245; H04L 67/10; G16H 20/00; G06N 20/00; G16H 10/60

(57) ABSTRACT

This patent discloses methods, systems, and a computer program product for remote analysis of synaptic docks of the human cranio-neural architecture, along with appropriate and complimentary services and incentives for combating drug abuse from a mobile (such as smartphones or tablets) or non-mobile device such as a computer, both of which utilize data networks to fine-tune decisions, along with user input data and previous behavior patterns, and other medical, social, weekly, and other parameter based data in deterministic and navigational processes. The methods and systems use primary and secondary data in real time to compute, evaluate, communicate, record, transact, including required features for the overall service.

### RELATED APPLICATIONS

None

### BACKGROUND & FIELD OF THE INVENTION

### Field of the Invention

The System, Method and computer program serves to make the process of drug rehabilitation and drug abuse abatement easier, more efficient, and more connected through multiple resources for the patients helps and needs and multiple databases and software systems to make analysis and predictions of the patients progress given the resources and different possible courses that the patient could take.

Multiple methods and services can be connected to an accessible application with an additional feature of predictive analytics on neuro-cranial structures and future symptoms or behaviour paths which can be accessed and utilized for a more precise service. Peer-to-Peer connection, data transfer between the network nodes for collecting data and evaluating it will prove for an enhanced experience.

### Background of the Invention

Modern age drug rehab and abatement procedures only involve physical and medical methods in centres to psychologically ‘correct’ victims, often done without care, treatment or exactment. Diagnosis, provisions, synopsis, and evaluation is all done in one school of thought and centre without any existing depth-ful analysis or retrospect and no personal care for specifics. Automation and formation of a digital network that could provide easy access and utilization of resources in a calculated manner for a quicker, more efficient and more successful process (as opposed to a statistically high amount of drug abusers falling back into their old lifestyle due to current ‘only’ rehabilitation methodologies).

Thus, the current human process of administration over rehabilitation can be largely controlled by accurate algorithms that provide the perfect combination of requirements. The current state of long, painful, psycho-inducing and body intensive method of abating drug abuse and educating everyone involved in a meaningful manner will be largely beneficial.

### SUMMARY

### Brief Summary of the Invention

The computer program product, system and method comprises of a centrally connected mobile application having a social networking, mapping, online ordering, medical-servicing, diagnosing, and AI based monitoring and advising (like Apple's SIRI) feature for the drug abuser to utilize during the complete abatement journey.

The application will have an easy access UI which is intuitive and containing access links to all network resources for appropriate use of the patient, along with weekly reports of actions, diagnosis, advised lifestyle and specifications, chat links to advisors, doctors, and similar minded people (previous drug addicts, current drug addicts), and synthesised map routes to ‘good’ spots, such as chemists, hospitals, police centres, or any other daily help spaces needed, with a enhanced helpline for each spots to the user.

This data set can be fine-tuned by user based weekly responses to NLP computed questionnaires (based on user diagnostics during app runtime ad advisor/doctor reports). This data is also kickstarted by extracting data from the user profile during application launch on local devices.

In real-time, as the procedure goes on, the patient can utilize any of the resources for their use, often at advisor discretion, to make the process more streamlined. Using mapping applications to get prescribed medicine or visit rehab centres for emergencies and so on, using delivery services for food, medicine and doctor orders, and using synthesized social media for positive motivation and ‘survivor’ stories can be really spearheading as a process (these 3 features are given in example among the many others).

The drug-reward system is a negative feedback loop that biologically rewards the abuser with more of dopamine for each drug intake exponentially. A key motivator of this system would be rewards of any sort for the material lacking in the personal life of the abuser, which is a statistically high cause for them to abuse drugs in the first place. Good track of treatment may be rewarded with such things, with appropriate funding.

With the network of services to use set, the question of resources required per user in total to be stockpiled, looked after, monitored, appropriately distributed, and a management network of funding companies (in reference or not in reference to the provided materials). This network is handled by all possible data being directly fed in to company directory for organized distribution of required goods and availability of services always.

Timely analysis of brain structure will be an integral role in advise towards medicine, resources, and so on. A cumulative scan with multiple methodologies will be taken from the patient, and will be extracted for image data. This data will be analysed using image processing, while accessing symptom data to find any correlation, and use synapse based analysis along with this image analysis data to predict future conditions, render present diagnosis, and link analysed condition to the situation of the full body (internal and external features down to the genetic composition).

### BRIEF DESCRIPTION OF DRAWINGS

FIG. 1: Overlaying the complete network of the application central, from user processes to all other aiding factors

FIG. 2: Describing the Data Structure of the τ functionality in detail in context of the total data structure for a brief understanding of synaptic analysis

FIG. 3: Showcasing the application UI (home screen only) to picture out the real-time product ‘face’

FIG. 4: Describing the Hardware System of the t functionality in detail in context of the total data structure for a brief understanding of the monitoring ‘node’

### SPECIFICATIONS—DETAILED DESCRIPTION

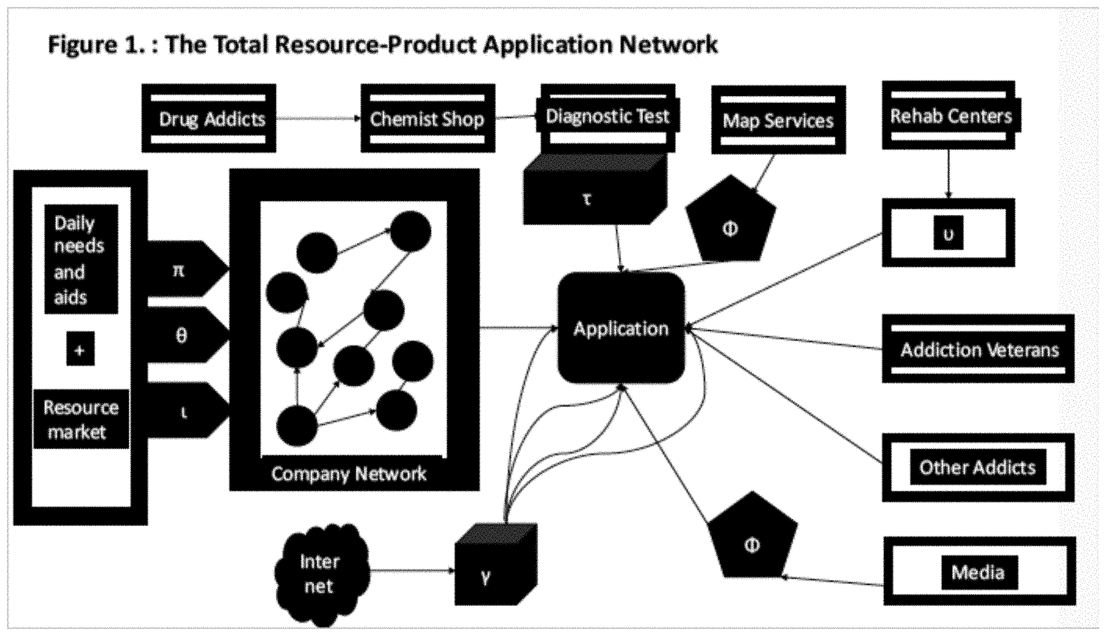

Referencing FIG. 1: The process of application launch starts with the drug abuser (user of application) logging into the downloaded application on any device and initially visiting a chemist shop for user entry based (profile and questionnaire) purchases (that are free and sponsored) which lead to a diagnostic test taken by the user. The materials used for the diagnostic test vary by users data, and some aspects are mandatory (such as the set of brain scans which are used in the neurocranial analysis in algorithm τ). This data after processing by sponsored diagnosis agencies are fed in specific to the users profile and is viewable by the user anytime.

Referencing FIG. 1: The application provides focused Map services from google APIs, where the focusing and filtering of appropriate and important places for the user (such as ER centers and so on) are highlighted and optimized for travel. The filtering, focusing, and data optimization is done by the 1 Φ unction. The application also has links for contact and registry into rehab centers, for emergencies and possible advised treatments. Psychological counseling can be offered as a first measure treatment to rehab as well by similar authorities and by advising/personal need.

Referencing FIG. 1: The application also has social media based links to previous drug addicts who escaped the vicious cycle and other addicts who are currently suffering. This special feature of connecting with these people may offer anecdotal advise or support or moral/ethical standpoints to each user. The features of the social media feature would be that of a standard one, like Instagram (with motivational picture sharing and filtering and monitoring for negative material regarding drugs, and chatting features to get in touch with).

Referencing FIG. 1: Φ function filtered media, news, reports and so on can also be viewed to suit the positive needs of the user, based on preferences, and user activity as well. These sources will provide needed support to the user in combating drug abuse.

Referencing FIG. 1: From the entirety of the internet, there will be an extracted database ‘γ’ of drug abuse related, drug symptom related, technical, rehab/medical center related data in a matrix for access into the application. This data is different from the map data, social media data and the rehab center or resource data. This data fed into the application is used for user info that is more streamlined to the focus of the application (more knowledge based and for AI to finetune user data in accordance to existing data).

Referencing FIG. 1: For resource distribution to users, typical ordering services like amazon's will be implemented with a stockpile that is monitored for quality. Periodic Monitoring of the resources, of the progress of the user, and of 3^{rd} party use and advise/medical help is also monitored (function t), along with advertisements of products that are catered to user's use (function θ, based on current AI based determination systems). These resources may be upped in value for a reward based system where progress in abating drugs is regarded with user's choice of commodities (optional by user) (function π) which may further boost recovery period.

Referencing FIG. 1: Forming the network of companies for resources, product and service based aspects can be done by finding conglomerates, single or any legal category of companies fitting in the agreement for the said part of the application, where the company has added shares and revenue from the sales of goods and services, relays monitoring and quality control and customer service information, and this can be fed into the application for actions to be taken.

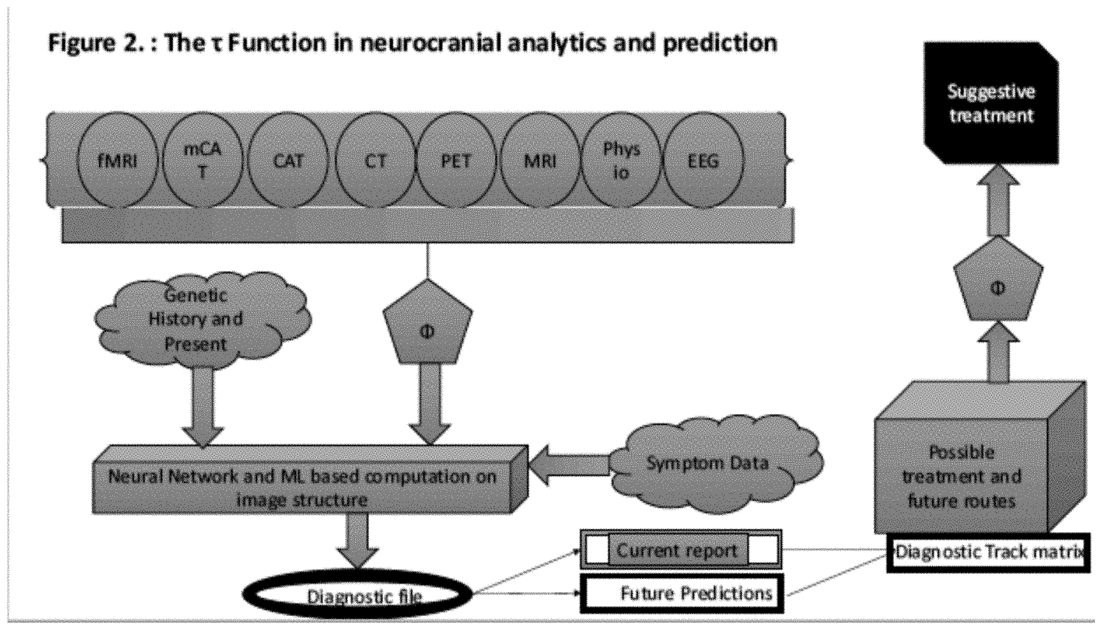

Referencing FIG. 2: The τ function as part of calculated time based diagnosis involves gathering scan data from a set of brain scans, from fMRI to EEG as shown in the figure. The information is extracted that is related to drugs physiological and/or chemical alterations. Using the user data entries of genetic history and genetic data of the person, the computation is performed to produce results.

Referencing FIG. 2: The computational function to provide deterministic analysis uses all drug related symptom data and tries to match the descriptions with image based understanding by NLP and uses recursive ML to render all extracted data against the network of gathered data and across all possible combinations of the data. The results are then computed, and stored in a single file having the analysis of the time based input set ready.

Referencing FIG. 2: The file comprises of 2 separate things: current conditional analysis (from NLP as a key feature) and future conditional predictions using ML based predictive analytics. This is cross referenced against the possible routes towards ailment to determine possibilities of other effects along the route and of other added benefits, which are then compiled against, and a best set is extracted and displayed as a UI on the ‘suggestive treatment’ area. The copies of result data is in user and related personnel applications. The application has a ‘login as’ feature where each category of affiliate can login for use.

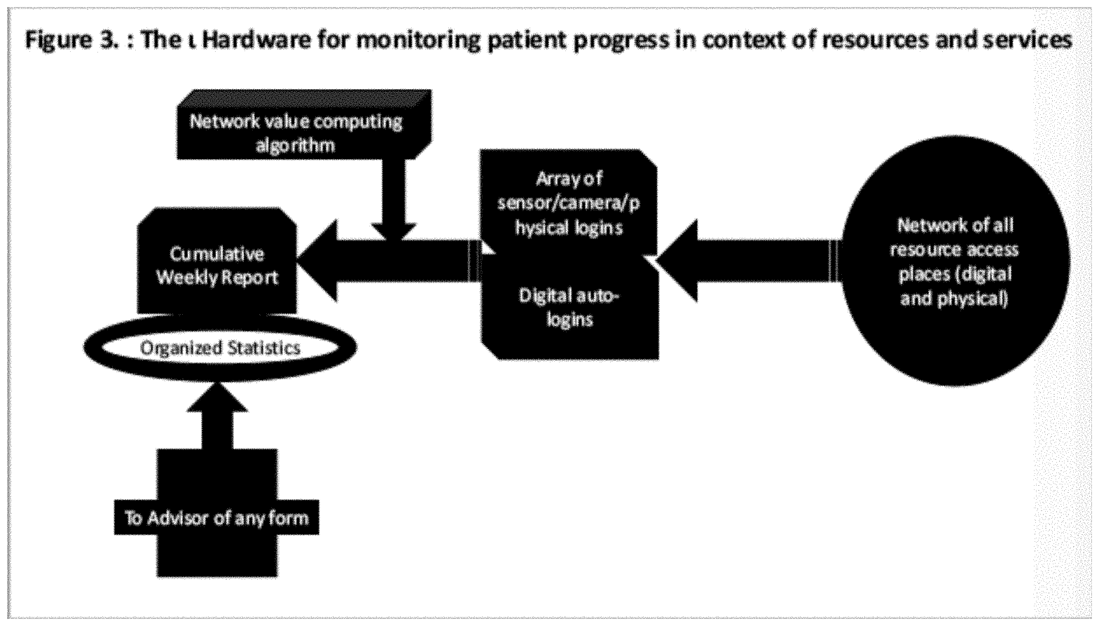

Referencing FIG. 3: The structure of monitoring resources, conditions of affiliated places in the main network in FIG. 1 and of all the types of users can be done by a generally linked monitoring system in the application network. Digital logins of any kind: purchases, app logins and so on are marked, and so are sensor based or camera base differences, such as rehab visits, stockpile expiry, location and amount and so on, which comprised of monitoring data.

Referencing FIG. 3: This data is then analyzed for fits in company decided parameters to compare it and form a weekly report of quality of monitoring, monitoring periods and so on. Further mathematical analysis of the same can produce some statistical analysis of the monitoring of all involved nodes in the network in FIG. 1. This is then relayed to the advisor in companies to adjust policies.

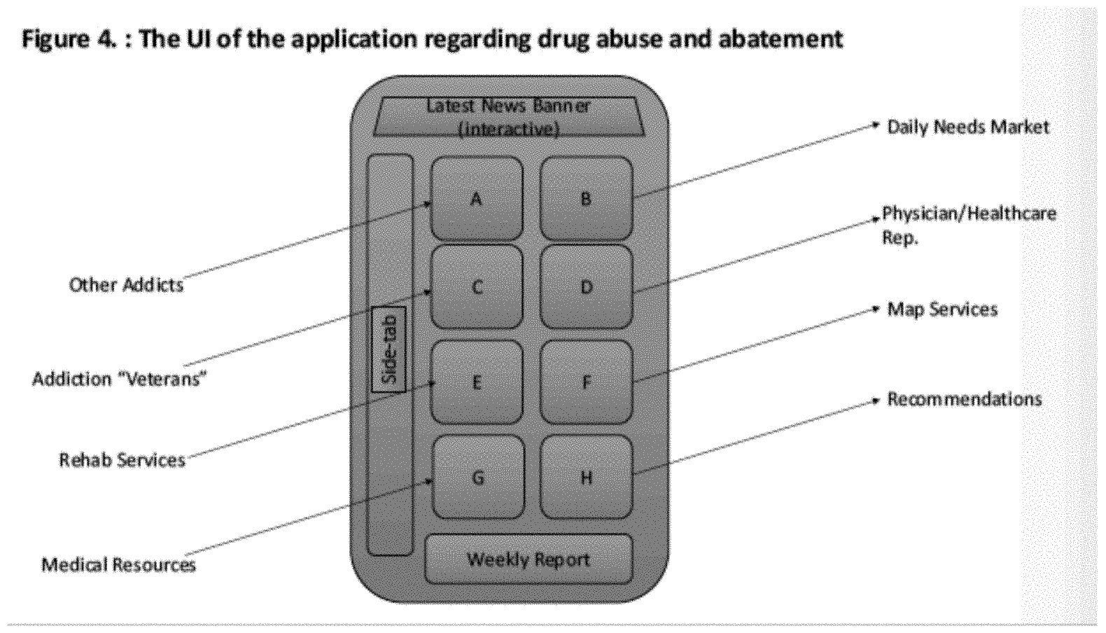

Referencing FIG. 4: The home screen of the application is seen where there is clear access to all the listed aspects and features for instant use. The side-tab can be used for note-making, quick personalized access or for quick routing to previous actions. Appropriate links to other sources/websites/or company leased features will be hosted in accordance.

Referencing FIG. 4: The home screen for different types of users (non-abusers) will be different. For advisors, the side-tab will host a list of linked patients for analysis and their respective histories in each of the respective application features, along with a weekly report and appointment scheduling feature.

Referencing FIG. 4: Regarding diagnostics and medical support, a similar fashion of layout to the advisors will follow but with more analyzed details and access to possible treatment routes.

Referencing FIG. 4: For monitoring agencies, more emphasis on monitored data in the t function will be displayed instead of possible treatment routes and appointment schedules and more analyzed user details.

While specific ideas and embodiments have been illustrated and described, numerous modifications come to mind without significantly departing from the spirit of the invention, and the scope of protection is only limited by the scope of the accompanying claims.

What is claimed is:

1. A system for analysis, predictions, evaluation, networking, end-to-end communication and sorting comprising of various processing algorithms to handle and communicate immense diagnosis, advising, user action, monitoring related past and current data,
mainly comprising of multiple cloud based services to store raw/semi-processed/processed data from all sources and mobile or non mobile device or non mobile devices and/or another device and mobile or non mobile device user file based search app client: and capable of receiving from a user or another device or system, a request to search or calculation of status, diagnosis, advisement determinations, the request comprising a keyword or multiple keywords or selections or values; based on receiving the request: retrieving, from storage databases, user status, advice, diagnostic, evaluation and deterministic analysis software engine comprising of;
a data device comprising a plurality of database entries each corresponding to a respective user data related asset (such as non calculated or calculated values or data or metadata or digitized content),
wherein each database entry comprises descriptive metadata associated with the respective advisement, diagnosis, monitoring and all user type(s) data related asset (such as non calculated or calculated values or data or metadata or digitized content);
comparing, using control circuitry, the keyword or multiple keywords or selections or values to the descriptive metadata associated with each of the plurality of database entries; identifying, based on the comparing, a subset of the plurality of database entries that are associated with the descriptive metadata that includes the keyword or multiple keywords or selections or values,
an application program interface to allow two way communication, interaction and data sharing between the computer program product and other relevant devices or systems,
wherein the subset of the database entries comprises database entries for advisement, diagnosis, monitoring and all user type(s) data related determinations;
and storing, in user or another device or system interaction metadata, the request;
receiving, from the user or another device or system, a selection of the advisement, diagnosis, monitoring and all user type(s) data related asset (such as non calculated or calculated values or data or metadata or digitized content); based on receiving the selection of the advisement, diagnosis, monitoring and all user type(s) data related asset (such as non calculated or calculated values or data or metadata or digitized content) storing, in user or another device or system interaction metadata associated with the request, an indication of the selection of the advisement, diagnosis, monitoring and all user type(s) data related asset; receiving, from the user or another device or system; generating a list of advisement, diagnosis, monitoring and all user type(s) data related determinations including the advisement, diagnosis, monitoring and all user type(s) data related asset (such as non calculated or calculated values or data or metadata or digitized content) and where in the advisement, diagnosis, monitoring and all user type(s) data related search app client comprises:
software to interact with and present data to a user via the user interface,
software to interact with and present data to another device or system interface,
software to retrieve data comprising at least one cloud based service reading from the another cloud based service associated with another device or system interface,
software to send the retrieved data from mobile or non mobile device to the advisement, diagnosis, monitoring and all user type(s) data related determinations search or calculation or calculator engine via the data network interface,
and wherein the system comprises:
wherein the at least one computer processor of at least one of: the mobile or non-mobile device or non mobile device, the advisement, diagnosis, monitoring and all user type(s) data related determinations search or calculation platform device, or the advisement, diagnosis, monitoring and all user type(s) data related determinations platform, is configured to: calculate the advisement, diagnosis, monitoring and all user type(s) data associated attributes;
and wherein the at least one processor is configured to send the advisement, diagnosis, monitoring and all user type(s) data associated attributes of the device to the advisement, diagnosis, monitoring and all user type(s) data related determinations search or deterministic evaluation or numero-text based analytical and network service feature based analytical engine via the data network interface, and receive advisement, diagnosis, monitoring and all user type(s) data related determinations data at the mobile device or non mobile device or non mobile device for the device, from the advisement, diagnosis, monitoring and all user type(s) data related determinations search or calculation or calculator engine via the data network interface, and display the advisement, diagnosis, monitoring and all user type(s) data related determinations data on the user or another device or system interface of the mobile or non-mobile device or non mobile device;
wherein the advisement, diagnosis, monitoring and all user type(s) data related determinations search or deterministic evaluation or numero-text based analytical and network service feature based analytical engine comprises wherein the at least one computer processor of the advisement, diagnosis, monitoring and all user type(s) data related determinations search or calculation platform device is configured to receive the derived or calculated data from the advisement, diagnosis, monitoring and all user type(s) data related determinations search or deterministic evaluation or numero-text based analytical and network service feature based analytical and network service feature based app client via the data network interface,
send the derived or calculated data to the advisement, diagnosis, monitoring and all user type(s) data related determinations process parameters determination subsystem computer program product via the data network interface, receive the advisement, diagnosis, monitoring and all user type(s) data related data from the advisement, diagnosis, monitoring and all user type(s) data related determinations process parameters determination subsystem via the data network interface, store the derived or calculated data and the advisement, diagnosis, monitoring and all user type(s) data related determinations data in the advisement, diagnosis, monitoring and all user type(s) data related determinations search or calculation database, and send the advisement, diagnosis, monitoring and all user type(s) data related determinations data to the advisement, diagnosis, monitoring and all user type(s) data related determinations search or deterministic evaluation or numero-text based analytical and network service feature based analytical and network service feature based app client via the data network interface.

2. The system according to claim 1 wherein: all user type(s) and the advisement, diagnosis, monitoring related determinations search or deterministic evaluation or numero-text based analytical and network service feature based analytical and network service feature based app is configured to read data from said another cloud service of said plurality of cloud services; wherein the cloud services are encrypted by a known key in the main function of the software.

3. The system according to claim 1 wherein all user type(s) and the advisement, diagnosis, monitoring related determinations search or deterministic evaluation or numero-text based analytical and network service feature based analytical engine comprises wherein the at least one computer processor of the all user type(s)data related determinations search or calculation platform device is configured to calculate the all user type(s)data related determinations from the received data.

4. The system according to claim 1 wherein: user type(s) and the advisement, diagnosis, monitoring data determinations search or deterministic evaluation or numero-text based analytical and network service feature based analytical and network service feature based app client further comprises wherein said at least one computer processor of the mobile device or non mobile device or non mobile device is configured to automatically communicate the all user type(s)data related determinations data to a pre-configured electronic address.

5. The system according to claim 1 wherein: user type(s) and the advisement, diagnosis, monitoring data determinations search or deterministic evaluation or numero-text based analytical and network service feature based analytical and network service feature based app client further comprises wherein said at least one computer processor of the mobile device or non mobile device or non mobile device is configured to display on the user or another device or system interface information regarding the all user type(s) related determinations data.

6. The system according to claim 1 wherein: user type(s) and the advisement, diagnosis, monitoring data determinations search or deterministic evaluation or numero-text based analytical and network service feature based analytical and network service feature based app client further comprises wherein said at least one computer processor of the mobile device or non mobile device or non mobile device is configured to allow the user or another device or system to send the all user type(s) related determinations data to a user or another device or system-specified electronic address to monitor, manage or control the device.

7. The system according to claim 1 wherein: user type(s) and the advisement, diagnosis, monitoring data determinations search or deterministic evaluation or numero-text based analytical and network service feature based analytical and network service feature based app client further comprises wherein said at least one computer processor of the mobile device or non mobile device or non mobile device is configured to display on the user or another device or system interface information regarding the user types related determinations data.

8. The system according to claim 1 wherein: complex mathematical and abstract computations with words and processing of adjunct source based and text/raw numeral based data to provide comprehensive diagnosis regarding drug abuse, physiochemical analyses, monitoring of usage and resources data and network based human-end interactions (companies, centers, services, staff) available purely by software systems, databases and data structures.

9. An all user type determinations client application computer program product embodied on a computer accessible medium configured to execute, on at least one computer processor of a mobile or non mobile device or non mobile device in communication or non communication with a all user type(s) related determinations search or calculation platform over a communications network, remotely obtaining all user type(s) related determinations data, comprising:
receiving, by the at least one computer processor, an interaction from a user or another device or system by communicating over data network;
calculating, by the at least one computer processor, all user type(s) related determinations or value;
incorporating an application program interface to allow two way communication, interaction and data sharing between the computer program product and other relevant devices or systems, wherein the at least one computer processor of the all user type(s) related determinations search or calculation platform device is configured to receive the derived or calculated data from the all user type(s) related determinations search or deterministic evaluation or numero-text based analytical and network service feature based analytical and network service feature based app client via the data network interface, send the derived or calculated data to the all user type(s) related determinations process parameters determination subsystem computer program product via the data network interface, receive the all user type(s) related determinations data from the all user type(s) related determinations process parameters determination subsystem via the data network interface, store the derived or calculated data and the all user type(s) related determinations data in the all user type(s) related determinations search or calculation database, and send all user related determinations data to the all user type(s) related determinations search or deterministic evaluation or numero-text based analytical and network service feature based analytical and network service feature based app client via the data network interface;
For responding, by the at least one computer processor, to the user or another device or system interaction by reading cloud based service readings from another cloud based service of said plurality of cloud based services; wherein the cloud based services connected to databases, or cloud based services connected to other users, and another cloud based service, other than a touchscreen, a keyboard, and a mouse;
forming, by the at least one computer processor, a all user type(s) related determinations search or calculation request by inserting, by the at least one computer processor, the cloud based service readings from the plurality of cloud based services into the all user type(s) related determinations search or calculation request; and
sending, by the at least one computer processor, the all user type(s) related determinations search or calculation request, over the communications network, to the all user type(s) related determinations search or calculation platform;
and receiving, by the at least one computer processor, all user type(s) related determinations data, from the communications network, in response to the all user type(s) related determinations search or calculation request; and wherein the method comprises: retrieving, by the at least one computer processor, data comprising the at least one cloud based service reading; and calculating, by the at least one computer processor, all user type(s) related determinations or values.

10. A method, comprising various processing algorithms to handle and communicate immense monitoring, diagnosing, advising, report based, management based, network node quality based, and user experience based data, on a mobile or non mobile device or non mobile devices and/or another device: capable of receiving from a user or another device or system, a request to search or calculation for all user type(s) (advisor, abuser, diagnoser, partners, resource distributers, managers, socialites) related determinations, the request comprising a keyword or multiple keywords or selections or values; based on receiving the request: retrieving, from storage circuitry, communicating with a data device comprising a plurality of database entries each corresponding to a respective all user type(s) (advisor, abuser, diagnoser, partners, resource distributers, managers, socialites) related asset (such as non calculated or calculated values or data or metadata or digitized content) wherein each database entry comprises descriptive metadata associated with the respective all user type(s) (advisor, abuser, diagnoser, partners, resource distributers, managers, socialites) related asset; comparing, using control circuitry, the keyword or multiple keywords or selections or value to the descriptive metadata associated with each of the plurality of database entries; identifying, based on the comparing, a subset of the plurality of database entries that are associated with the descriptive metadata that includes the keyword or multiple keywords or selections or values,
wherein the subset of the database entries comprises database entries for all user type(s) (advisor, abuser, diagnoser, partners, resource distributers, managers, socialites) related determinations; and storing, in user or another device or system interaction metadata, the request; receiving, from the user or another device or system, a selection of the all user type(s) (advisor, abuser, diagnoser, partners, resource distributers, managers, socialites) related asset; based on receiving the selection of the all user type(s) (advisor, abuser, diagnoser, partners, resource distributers, managers, socialites) related asset (such as non calculated or calculated values or data or metadata or digitized content) storing, in user or another device or system interaction metadata associated with the request, an indication of the selection of the all user type(s) (advisor, abuser, diagnoser, partners, resource distributers, managers, socialites) related asset; receiving, from the user or another device or system; generating a list of all user type(s) (advisor, abuser, diagnoser, partners, resource distributers, managers, socialites) related determinations including the all user type(s) (advisor, abuser, diagnoser, partners, resource distributers, managers, socialites) related asset (such as non calculated or calculated values or data or metadata or digitized content) and wherein the system comprises: wherein the at least one computer processor of at least one of: the mobile or non-mobile device or non mobile device, the all user type(s) (advisor, abuser, diagnoser, partners, resource distributers, managers, socialites) related determinations search or calculation platform device, or the all user type(s) (advisor, abuser, diagnoser, partners, resource distributers, managers, socialites) related determinations platform, is configured to:
calculate the all user type(s) (advisor, abuser, diagnoser, partners, resource distributers, managers, socialites) related or associated attributes; and wherein the at least one processor is configured to send the all user type(s) (advisor, abuser, diagnoser, partners, resource distributers, managers, socialites) related or associated attributes of the device to the all user type(s) (advisor, abuser, diagnoser, partners, resource distributers, managers, socialites) related determinations search or deterministic evaluation or numero-text based analytical and network service feature based analytical engine via the data network interface, and receive all user type(s) (advisor, abuser, diagnoser, partners, resource distributers, managers, socialites) related determinations data at the mobile device or non mobile device or non mobile device for the device, from the all user type(s) (advisor, abuser, diagnoser, partners, resource distributers, managers, socialites) related determinations search or deterministic evaluation or numero-text based analytical and network service feature based analytical engine via the data network interface, and display the all user type(s) (advisor, abuser, diagnoser, partners, resource distributers, managers, socialites) related determinations data on the user or another device or system interface of the mobile or non-mobile device or non mobile device;
wherein the all user type(s) (advisor, abuser, diagnoser, partners, resource distributers, managers, socialites) related determinations search or deterministic evaluation or numero-text based analytical and network service feature based analytical engine comprises wherein the at least one computer processor of the all user type(s) (advisor, abuser, diagnoser, partners, resource distributers, managers, socialites) related determinations search or calculation platform device is configured to receive the derived or calculated data from the all user type(s) (advisor, abuser, diagnoser, partners, resource distributers, managers, socialites) related determinations search or deterministic evaluation or numero-text based analytical and network service feature based analytical app client via the data network interface, send the derived or calculated data to the all user type(s) (advisor, abuser, diagnoser, partners, resource distributers, managers, socialites) related determinations process parameters determination subsystem computer program product via the data network interface, receive the all user type(s) (advisor, abuser, diagnoser, partners, resource distributers, managers, socialites) related determinations data from the all user type(s) (advisor, abuser, diagnoser, partners, resource distributers, managers, socialites) related determinations process parameters determination subsystem via the data network interface, store the derived or calculated data and the all user type(s) (advisor, abuser, diagnoser, partners, resource distributers, managers, socialites) related determinations data in the all user type(s) (advisor, abuser, diagnoser, partners, resource distributers, managers, socialites) related determinations search or calculation database, and send the all user type(s) (advisor, abuser, diagnoser, partners, resource distributers, managers, socialites) related determinations data to the all user type(s) (advisor, abuser, diagnoser, partners, resource distributers, managers, socialites) related determinations search or deterministic evaluation or numero-text based analytical and network service feature based analytical app client via the data network interface.

11. A set of method descriptions in accordance to claim 8 where:
i. The method according to claim 8 wherein: the all user type(s) (advisor, abuser, diagnoser, partners, resource distributers, managers, socialites) related determinations search or deterministic evaluation or numero-text based analytical and network service feature based analytical app is configured to read data from said another cloud based service of said plurality of cloud based services; wherein the cloud based services connected to databases and 3rd party sources, or cloud based services connected to other users.
ii. The method according to claim 8 wherein at least one computer processor of the all user type(s) (advisor, abuser, diagnoser, partners, resource distributers, managers, socialites) related determinations search or calculation platform device is configured to calculate the all user type(s) (advisor, abuser, diagnoser, partners, resource distributers, managers, socialites) related determinations from the received data.
iii. The method according to claim 8 wherein: the all user type(s) (advisor, abuser, diagnoser, partners, resource distributers, managers, socialites) related determinations search or deterministic evaluation or numero-text based analytical and network service feature based analytical app client of the mobile device or non mobile device or non mobile device is configured to automatically communicate the all user type(s) (advisor, abuser, diagnoser, partners, resource distributers, managers, socialites) related determinations data to a pre-configured electronic address.
iv. The method according to claim 8 wherein: the all user type(s) (advisor, abuser, diagnoser, partners, resource distributers, managers, socialites) related determinations search or deterministic evaluation or numero-text based analytical and network service feature based analytical app client of the mobile device or non mobile device or non mobile device is configured to allow the user or another device or system to send the all user type(s) (advisor, abuser, diagnoser, partners, resource distributers, managers, socialites) related determinations data to a user or another device or system-specified electronic address to monitor, manage or control the device.
v. The method according to claim 8 wherein: the all user type(s) (advisor, abuser, diagnoser, partners, resource distributers, managers, socialites) related determinations search or deterministic evaluation or numero-text based analytical and network service feature based analytical app client of the mobile device or non mobile device or non mobile device is configured to display on the user or another device or system interface information regarding the all user type(s) (advisor, abuser, diagnoser, partners, resource distributers, managers, socialites) related determinations data.

12. Software based modifications of the computer program product incorporating:
i. A computer program product involving: multiple layers and dimensions of sorting, classifying, pre-processing and associating of initial data and suggestive data along with other related inputs along the data flow of the software algorithm functionality.
ii. A computer program product involving: complex flows (with switch-case, split function, multi-storage, statistical and NLP (enhanced with ML) based standard algorithms) and invoking of data along the activation and result display processes of the software system, wherein data is processed to provide various aptitudes of understanding monitoring, user, diagnostic, analytical data in context of multiple fields.
iii. A computer program product and method involving a network system of suppliers, buyers, monitoring agencies, software analyzing algorithms, data centers, and service chains that interact with each other by wireless data transfer regarding specific statuses of related user type(s).

13. A set of methods and systems using growing technical aspects in software to enhance proficiency by:
i. A method of which an integral part is: the image processing algorithm clients and text to image comparisons for deterministic evaluation regarding the complete computer program product and related nodes of users, network components of hardware nature, and managerial systems.
ii. A system of which subsets can: predict events and render present conditions based on processed parameters specific to multiple event and scenario rundowns in medical prescriptive paths and diagnosis.
iii. A system containing an intuitive and multifeatured access based UI for all types of users with special features in each client login setting for smooth operation, efficient management, and for collection of the most optimal set of data for future and current evaluation schemes of multiple natures; medical, advisory and others.
iv. A system having an optimized conglomerate of resource givers, managers, and related corporate aid that adequately functions in providing and managing each task related for a network of users on a regular basis.
v. A method of which a primary function is: a monitoring agenda software protocol based on Internet of Things in the application central network, where appropriate data from user type(s), resources, stocks, managers, and results are gathered on set parameters and specifications to determining the future set of monitoring and policy or evaluation based actions.
While certain aspects of the disclosure are presented below in certain claim forms, the inventor contemplates the various aspects of the disclosure in any number of claim forms. For example, while only one aspect of the disclosure is recited as a means-plus-function claim under 35 U.S.C. .sctn.112, 6, other aspects may likewise be embodied as a means-plus function claim, or in other forms, such as being embodied in a computer-readable medium. (Any claims intended to be treated under 35 U.S.C. .sctn.112, 6 will begin with the words “means for”.) Accordingly, the applicant reserves the right to add additional claims after filing the application to pursue such additional claim forms for other aspects of the disclosure.
